(19) United States
(12) Patent Application Publication
(10) Pub. No.: US 2022/0000581 A1
(43) Pub. Date: Jan. 6, 2022

(54) ANTI-AEROSOLIZING TENT

(71) Applicants: Jonathan NOVER, Brooklyn, NY (US); Michael OLSHANSKY, Brooklyn, NY (US); James SMALDONE, Westfield, NJ (US)

(72) Inventors: Jonathan NOVER, Brooklyn, NY (US); Michael OLSHANSKY, Brooklyn, NY (US); James SMALDONE, Westfield, NJ (US)

(21) Appl. No.: 16/921,597

(22) Filed: Jul. 6, 2020

(51) Int. Cl. A61B 90/40

(52) U.S. Cl. CPC A61B 90/40; A61B 2090/571

(57) ABSTRACT

A shielding device movable between a folded configuration, in which the shielding device is stored, and an expanded configuration, in which the shielding device is configured to be positioned over a patient to prevent a spread of aerosolized particles. The shielding device includes a flexible material configured to be movable between the folded configuration and the expanded configuration to form a tent portion, a wall of the tent portion defining an interior space within which the patient is received in an operative configuration and a tensile frame including a plurality of struts, each connected to a portion of the flexible material and biased toward the expanded configuration, in which each of the struts provide a tension across the portion of the flexible material to which it is connected, the struts being deformable toward the folded configuration so that portions of the flexible material may be folded against itself.

### BACKGROUND

Intubation is a medical procedure in which a tube is inserted through a mouth and into a trachea to open a patient's airway. Intubation is often performed prior to surgery or during emergencies so that the patient may be placed on a ventilator to assist with breathing during anesthesia, sedation or severe illness. Most hospital Emergency Departments (ED) have selected rooms for intubation—rooms that are used for critical life saving interventions. These rooms, however, are generally few in comparison to the overall number of rooms in an ED, as the volume of critically ill patients presenting to an ED at any given time is low in comparison to the majority volume of patients that are moderate to low acuity.

During a disease outbreak such as, for example, the COVID-19 surge, EDs nationwide are required to perform life-saving interventions in all areas of the ED. The surge of high acuity patients in a short period of time requires ED healthcare workers to perform intubations in any space available. The ED healthcare worker is most vulnerable to transmissions of viral infectious diseases during aerosol-generating procedures such as intubations. The virus is able to transfer via droplets, which spread through the air. Intubation shields for combatting the transfer of infectious diseases via aerosolization have recently been developed, and are formed of a rigid material (e.g., plexiglass) that is shaped as a four-sided box configured to be placed over a patient's head. These intubation shields, however, are impractical during an outbreak requiring the treatment of a high volume of patients in a short period of time, as they require sanitization between uses, are cumbersome due to its size, are heavy, have openings therefore exposing the healthcare worker, and are generally only stored in designated intubation areas.

### SUMMARY

Some exemplary embodiments are related to a shielding device movable between a folded configuration, in which the shielding device is stored, and an expanded configuration, in which the shielding device is configured to be positioned over a patient to prevent a spread of aerosolized particles. The shielding device includes a flexible material configured to be movable between the folded configuration and the expanded configuration, in which the flexible material is tensioned to form a tent portion, a wall of the tent portion defining an interior space within which the patient is received in an operative configuration and a tensile frame including a plurality of struts, each of the struts connected to a portion of the flexible material and biased toward the expanded configuration, in which each of the struts provide a tension across the portion of the flexible material to which it is connected, the struts being deformable toward the folded configuration so that portions of the flexible material may be folded against itself.

Other exemplary embodiments are related to a system for providing a portable barrier between a patient and a healthcare worker. The system includes a shielding device including a flexible material movable between a folded configuration and an expanded configuration via a tensile frame connected thereto, the tensile frame including a plurality of struts connected to the flexible member, each of the struts biased toward a tensioning configuration for tensioning a portion of the flexible material toward the expanded configuration, in which the flexible material defines a tent portion configured to be positioned over a patient to cover the patient, and each of the struts being deformable toward a collapsed configuration in which portions of the flexible material are folded against itself in the folded configuration, and a packaging sized and shaped to receive the shielding device therewithin, in the folded configuration, the struts constrained toward the collapsed configuration via an interior surface of the packaging when the shielding device is received therewithin and reverting to the tensioning configuration to move the flexible material toward the expanded configuration as the shielding device is removed from within the packaging.

### BRIEF DESCRIPTIONS OF DRAWINGS

FIG. 1 shows a perspective view of a system according to various exemplary embodiments.

FIG. 2 shows a perspective view of a shielding device according to various exemplary embodiments.

FIG. 3 shows a plan view of a first end of the shielding device according to various exemplary embodiments.

FIG. 4 shows a perspective view of a portion of a shielding device according to various exemplary embodiments.

FIG. 5 shows a top view of an exemplary shielding device that includes a chest compression access area according to various exemplary embodiments.

FIG. 6 shows a side view of an exemplary shielding device that includes a chest compression access area according to various exemplary embodiments.

### DETAILED DESCRIPTION

The exemplary embodiments may be further understood with reference to the following description and the related appended drawings, wherein like elements are provided with the same reference numerals. The exemplary embodiments relate to a medical shielding device and, in particular, relate to a device configured to shield and protect healthcare workers from aerosolized particles resulting during an aerosol-generating procedure such as intubation. Exemplary embodiments describe a shielding device configured as a pop-up tent sized, shaped and configured to be positioned over a patient during an aerosol-generating procedure when in an expanded configuration. As will be described in greater detail below, the shielding device may be sized, for example, in a full body size, a half body size or a neck and head size. The shielding device is comprised of a transparent, flexible material and a tensile frame including a plurality of deformable struts for moving the transparent material between a folded configuration and the popped-up expanded configuration. In particular, the shielding device is stored in the folded configuration within a packaging so that, as the shielding device is removed from the packaging, the shielding device is moved toward the expanded configuration. Thus, the shielding device, in the folded configuration, is portable so that the shielding device can be easily transported and utilized in any area of a hospital and may be disposed of upon completion of use. It will be understood by those of skill in the art that although the exemplary embodiments are described with respect to an intubation procedure, the shielding device of the exemplary embodiments may be used for any of a variety of aerosol-generating procedures including, but not limited to, intubation, extubation, ventilation, scoping, aspiration, airway suctioning, etc., or in any of a variety of other circumstances where a barrier between patient and healthcare worker may be necessary.

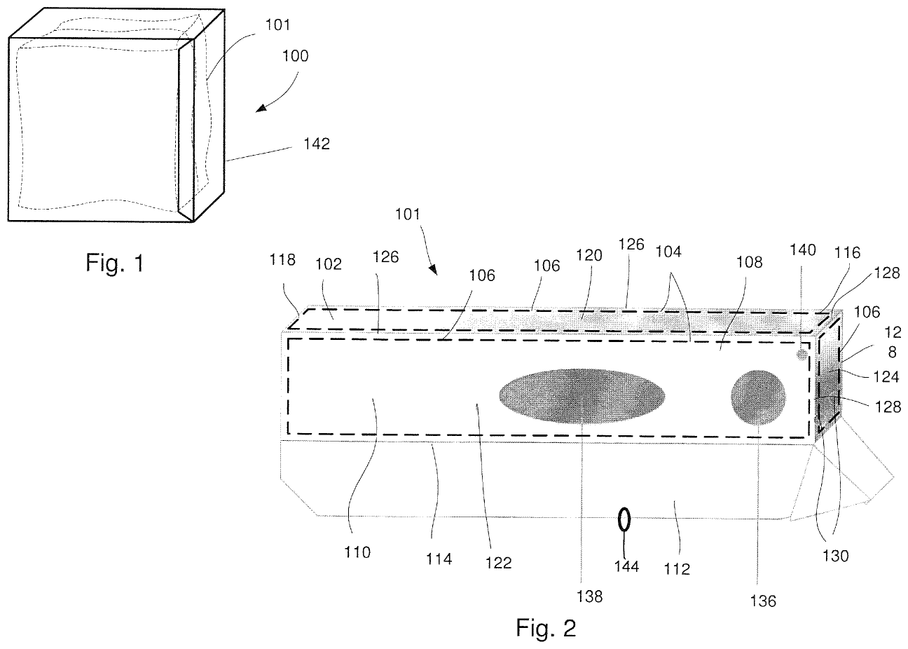

According to one exemplary embodiment, as shown in FIGS. 1-4, a system 100 comprises a shielding device 101 configured to be stored within a packaging 142, when in a folded configuration, and movable toward an expanded configuration as the shielding device 101 is removed from the packaging. The shielding device 101 is comprised of a transparent, flexible material 102 connected to a tensile frame 104 including a plurality of deformable struts 106 so that the flexible material 102 is movable between a folded configuration and an expanded configuration. In the folded configuration, the deformable struts 106 are deformed to collapse the tensile frame 104 and fold the flexible material 102 so that the shielding device 101 may be stored within the packaging 142 which facilitates portability of the shielding device 101. As shown in FIG. 1, an interior surface of the packaging 142 constrains the deformable struts 106 toward the collapsed, folded configuration so that, as the shielding device 101 is removed from the packaging, the deformable struts 106 revert toward a biased configuration so that the flexible material 102 is unfolded and moved toward the expanded configuration, which includes a tented portion 108. In other exemplary embodiments, when in the folded position, the shielding device 101 may also include another manner of maintaining the shielding device 101 in the folded position, e.g., a Velcro strap, a plastic strap/wrap, etc. In these exemplary embodiments, opening the package 142, alone, will not cause the shielding device 101 to open. A healthcare worker removing the strap or wrap is the last mechanism to open the shielding device 101. The tented portion 108 is configured to be positioned over a patient during an aerosol-generating procedure (e.g., intubation) such that the patient is received within an interior space defined by a wall 110 of the tented portion 108. The wall 110 acts as a barrier between the patient and the healthcare worker to prevent the transmission of disease via the aerosolized particles. As will be described in further detail below, the wall 110 includes a pair of arm holes 130 via which a physician or other healthcare worker performs the aerosol-generating procedure along with one or more entry ports via which intubation instruments or other medical devices may be inserted to treat the patient within the interior space of the tented portion 108. The shielding device 101 may also include a skirted portion 112 extending from contacting edges 114 of the wall 110 of the tented portion 108 to be draped over and/or attached to a hospital bed, stretcher, gurney or other surface on which the patient lies.

As described above, the flexible material 102 is transparent so that, when the tented portion 108 is placed over a patient, the patient is visible through the flexible material 102. Thus, a physician or other healthcare worker is able to perform the intubation, or other medical procedure, by visualizing the patient through the flexible material 102 of the tented portion 108. The flexible material 102 is sized and shaped so that, when in the expanded configuration, the flexible material 102 is tensioned via the tensile frame 104, as will be described in further detail below, to define the tented portion 108 and the skirted portion 112. The flexible material 102 is also configured to be foldable so that, in the folded configuration, the shielding device 101 is substantially flattened and reduced in size to be stored within the packaging 142. The packaging 142 may be configured as, for example, a bag or a box. The flexible material 102 may include a clear plastic material such as, for example, polyethylene and/or polypropylene.

As shown in FIG. 2, the tented portion 108 extends longitudinally from a first end 116 to a second end 118 so that the tented portion 108 is positionable over a length of the patient to cover and enclose at least a portion of a length of the patient including the patient's head, in an operative position. In one embodiment, the tented portion 108 is sized and shaped so that the interior space defined via the wall 110 of the tented portion 108 is substantially rectangularly shaped. In this embodiment, the wall 110 is further defined via an anterior surface 120 and a pair of lateral surfaces 122, each of which extend from the first end 116 to the second end 118. The pair of lateral surfaces 122 extend from opposing lateral edges 126 of the anterior surface 120 so that the lateral surfaces 122 extend along lateral sides of the patient and the anterior surface 120 extends along an anterior side of the patient in the operative position. The wall 110 is also defined via a superior surface 124 at the first end 116, which connects superior edges 128 of each of the anterior surface 120 and the lateral surfaces 122 to define the substantially rectangular interior space. In the operative position, the superior surface 124 extends proximate the patient's head, between, for example, the patient's head and the healthcare worker performing the intubation.

In one embodiment, a length of the tented portion 108 (e.g., a distance between the first and second ends 116, 118) may substantially correspond to a length of the hospital bed, stretcher, gurney or other surface on which the patient lies, while a width of the tented portion 108 (e.g., a distance between the pair of lateral surfaces 122) substantially corresponds to a width of the hospital bed, stretcher, gurney or other surface. For example, the tented portion 108 may be sized and shaped to accommodate a standard stretcher having a length of 90 inches and a width of 40 inches. It would be understood by those of skill in the art, however, that the length and width of the tented portion 108 may be varied, as desired, so long as the tented portion 108 is sized, shaped and configured to cover at least a portion of a length of the patient including the patient's head, to reduce a risk of transmission of disease via aerosolized particles. In one particular example, the tented portion 108 may be sized and shaped for full body coverage (e.g., coverage of the patient from head to toe) while in another example, the tented portion 108 may be sized and shape for half body coverage (e.g., coverage of the patient from head to waist). In other exemplary embodiments, the tented portion 108 may be sized to fit over the head and neck of the patient.

It will also be understood by those of skill in the art that while the exemplary embodiments show and describe the wall 110 of the tented portion 108 as defining a substantially rectangular shaped, the tented portion 108 may define any of a variety of shapes of interior spaces so long as the tented portion 108 is configured to be positioned over and cover a desired length of the patient during, for example, an aerosolizing procedure. In another embodiment, for example, the wall 110 may define an arched tunnel shape extending longitudinally from the first end 116 to the distal end 118, the first end 116 closed via a superior surface.

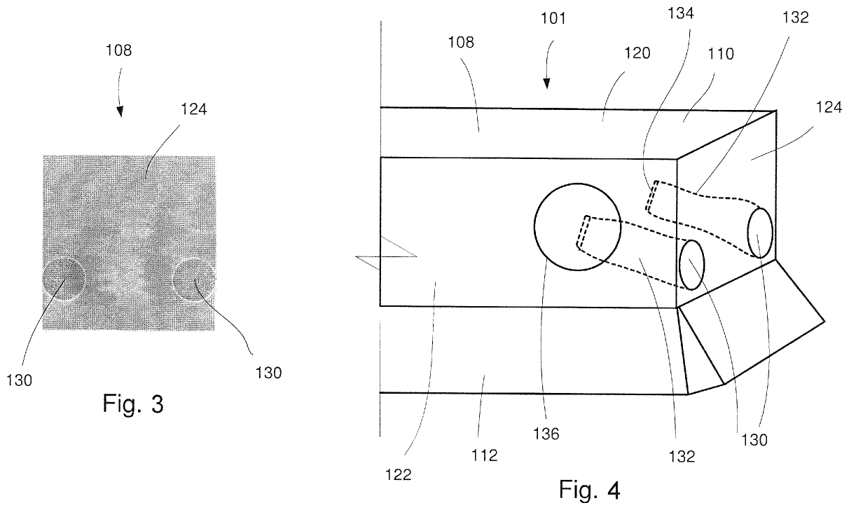

As described above, the healthcare worker may perform a medical procedure via the pair of arm holes 130 extending through the wall 110. As shown in FIG. 3, the arm holes 130 may, in one embodiment, extend through the superior surface 124 of the wall 110 so that, when the tented portion 108 is in the operative position, the healthcare worker has access to the patient's head to perform, for example, the intubation procedure. As shown in FIG. 4, each of the aim holes 130 may include sleeves 132 extending into the interior space of the tented portion 108 from the arm holes 130 to a ribbed end 134. The ribbed ends 134 ensure that a seal is present when the healthcare worker's arms are inserted through the sleeve and into the interior space of the tented portion 108. The sleeves 132 may be formed of the same material as the flexible material 102 of the tented portion 108.

The shielding device 101 also includes one or more entry ports or holes via which instruments or medical devices may be inserted through the wall 110 and into the interior space defined therewithin. In one embodiment, the wall 110 includes a first entry hole 136 extending through a portion of the wall 110 which, in the operative position, is proximate the patient's head. Instruments facilitating intubation and/or ventilation such as, for example, bag masks or tubing, may be inserted through the first entry hole 136. The wall 110 may also include a second entry hole 138 extending through a portion of the wall 110 corresponding in position to, for example, one of a chest, limb, abdomen and/or groin of a patient when the tent portion 108 is in the operative position. The second entry hole 138 thus extends through a portion of the wall 110 that is in an inferior direction relative the first entry hole 136. The second entry hole 138 may be configured to receive, for example, intravenous lines or other medical instruments. In one embodiment, the first and/or second entry holes 136, 138 extend through one of the lateral surfaces 122. Similarly to the arm holes 130, the first and/or second entry holes 136, 138 may also include a ribbing or other seal (e.g., valve) so that the interior space defined via the tent portion 108 remains sealed to an exterior of the tent portion 108 even when instruments are inserted through the holes 136, 138, when the tent portion 108 is in the operative position.

The shielding device 101 may also include a suction port 140 extending through a portion of the wall 110. The suction portion 140 is configured to be connected to a suction tube for creating a negative pressure system within the tent portion 108. The suction portion 140 may include, for example, a barb or other connector for connecting the tent portion 108 to the suction tube. In one embodiment, the suction port 140 extends through a portion of the wall 110 at the first end 116 of the tent portion 108.

The skirted portion 112 of the flexible material 102 extends from contacting edges 114 of the wall 110, which come into contact with the hospital bed, stretcher, gurney or other surface on which the patient lies. In one embodiment, in the operative position, the skirted portion 112 is configured to be draped over edges of the hospital bed, stretcher, gurney or other surface and includes a plurality of clips 144 configured to be harnessed to, for example, side rails of the hospital bed, stretcher or gurney. In the example of FIG. 2 only a single clip is shown for illustrative purposes, but those skilled in the art will understand that any number of clips may be used to secure the tent portion to the bed, stretcher, gurney, etc. The clips 144 fix the tent portion 108 over the patient and ensure that the tent portion 108 remains steady during the intubation procedure. Although the exemplary embodiments describe and show the skirted portion 112 with clips 144, it will be understood by those of skill in the art that, the tented portion 108 may be attached to the hospital bed, stretcher or gurney using any of a variety of attachment mechanisms including, but not limited to, adjustable straps.

In another example, the skirted portion 112 may include an elastic type material that may be stretched over and secured to the hospital bed, stretcher, gurney, etc. in a manner similar to the operation of a fitted sheet. In other exemplary embodiments, the shielding device 101 may include one or more straps that can be secured to the patient and/or the bed, stretcher or gurney to hold the tented portion 108 in place. This embodiment may be particularly useful for the head and neck size of the shielding device 101 because there is less area to secure the shielding device 101 to the bed. For example, a first end of the strap may be connected to a first part of the frame 104 and a second end of the strap may be connected to a second part of the frame 104. The strap may be secured behind the patient, e.g., around the head, neck, shoulders, or arms, in a manner that secures the tented portion 108 such that it covers the head and neck of the patient. Those skilled in the art will understand that this is only an example and any size of the shielding device 101, e.g., full length, half length, head and neck, may use the strap(s) to secure the shielding device 101. Similarly, any of the securing methods described herein, draping, clips, fitted, straps may be applied to any of the sizes of the shielding device 101.

As described above, the flexible material 102 is connected to deformable struts 106 of the tensile frame 104 so that the flexible material 102 is movable from the folded configuration to the expanded configuration, in which the flexible material 102 is tensioned to define the tented portion 108, as the shielding device 101 is removed from the packaging. The tensile frame 104 includes a plurality of deformable struts 106, each deformable strut 106 biased toward the expanded configuration and connected to a portion of the wall 110 so that, in the expanded configuration, the deformable struts 106 revert to their biased configuration to provide tension along portions of the flexible material 102 and define the tented portion 108. The deformable struts 106 of the tensile frame 104 may be formed of a metal, plastic or composite material.

Each surface 120, 122, 124 of the wall 110 is connected to one or more of the deformable struts 106 so that the deformable struts 106 along each of the surfaces 120, 122, 124 together form the tensile frame 104. In one example, a single deformable strut 106 is connected to one or more of the surfaces 120, 122, 124 so that the single deformable strut 106 extends along peripheral edges thereof to form a substantially a closed loop. In another example, multiple deformable struts 106 are connected to one or more of the surfaces 120, 122, 124 so that the multiple deformable struts 106 together extend along the peripheral edges of the one or more surfaces 120, 122, 124. Where the surfaces 120, 122, 124 include more than one deformable strut 106, positions of ends of the struts 106 correspond to a position of a corner of the surfaces 120, 122, 124 to facilitate a folding of the flexible material 102 at the corners of the surfaces 120 122, 124.

In one embodiment, in the folded configuration, the surfaces 120, 122, 124 and the skirted portion 112 of the flexible material 102 may be folded against one another so that the flexible material 102 is substantially folded into a flattened configuration. The deformable struts 106 are deformed to further fold the flexible material 102 into a compact size (e.g., having dimensions smaller than dimensions of the anterior surface 120 and/or the lateral surfaces 122). The deformable struts 106 may be deformed by, for example, coiling or otherwise bending each of the deformable struts 106 so that the flexible material 102 is further folded against itself. In the folded configuration, the shielding device 101 is stored within the packaging 142 so that the deformable struts 106 are constrained toward this folded configuration via an interior of the packaging. As the shielding device 101 is removed from the packing, however, the deformable struts 106 revert toward their biased configuration, tensioning and expanding the flexible material 102 to define the tent portion 108 and the skirt portion 112.

Once the shielding device 101 is in the expanded configuration, the tent portion 108 is positioned over a length of the patient, covering the patient's head so that the patient's head and at least a portion of a length of the patient is enclosed within the interior space of the tent portion 108. In one embodiment, the tent portion 108 is affixed in the operative position via clips 144 along the skirt portion 112, which is draped over edges of the hospital bed, stretcher, or gurney on which the patient lies. The physician or other healthcare worker may perform an intubation or other medical procedure via the aim holes 130 extending through the superior surface 124 the wall 110 of the tent portion 110. As described above, required instruments are inserted into the interior space of the tent portion 108 via the entry holes 136, 138.

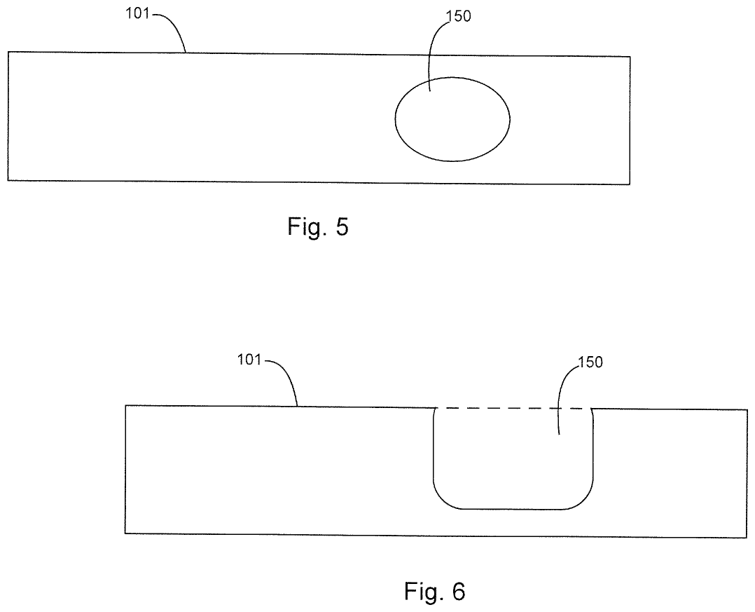

FIG. 5 shows a top view of an exemplary shielding device 101 that includes a chest compression access area 150 according to various exemplary embodiments. The need to perform chest compressions may arise before, during, or after the intubation procedure takes place. In some exemplary embodiments, the shielding device 101 allows clinicians the ability to perform chest compressions. The chest compression access area 150 will align with the chest cavity of the patient. It will be understood that the head and neck size shielding device 101 may not include the chest compression access area 150 because this size does not cover the chest of the patient. However, any size shielding device 101 that covers the chest of the patient may include the chest compression access area 150. The chest compression access area 150 may be a collapsible plastic material that allows the practitioner to touch the chest of the patient without compromising the integrity of the tent portion 108 or exposing the practitioner to aerosolizing particles. Thus, the practitioner may place their hands over the chest compression access area 150 and touch the chest of the patient to perform compressions through the collapsible plastic material.

FIG. 6 shows a side view of an exemplary shielding device 101 that includes a chest compression access area 150 according to various exemplary embodiments. In this exemplary embodiment, it may be considered that the practitioner is performing chest compressions and the collapsible plastic material of the chest compression access area 150 has been pushed down to the chest of the patient.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

What is claimed is:

1. A shielding device movable between a folded configuration, in which the shielding device is stored, and an expanded configuration, in which the shielding device is configured to be positioned over a patient to prevent a spread of aerosolized particles, the shielding device comprising:
a flexible material configured to be movable between the folded configuration and the expanded configuration, in which the flexible material is tensioned to form a tent portion, a wall of the tent portion defining an interior space within which the patient is received in an operative configuration; and
a tensile frame including a plurality of struts, each of the struts connected to a portion of the flexible material and biased toward the expanded configuration, in which each of the struts provide a tension across the portion of the flexible material to which it is connected, the struts being deformable toward the folded configuration so that portions of the flexible material may be folded against itself.

2. The shielding device of claim 1, wherein the flexible material is a clear plastic material.

3. The shielding device of claim 1, wherein the interior space defined via the tent portion is substantially rectangular.

4. The shielding device of claim 1, further comprising a pair of arm holes extending through a portion of the wall of the tent portion.

5. The shielding device of claim 4, wherein the pair of arm holes extend through a superior surface of the wall configured to positioned proximate a patient's head in the operative configuration.

6. The shielding device of claim 4, further comprising a pair of sleeves, each of the sleeves extending into the interior space defined via the tent portion from a corresponding one of the arm holes to a ribbed end configured to seal the interior space from an exterior of the of the tent portion when an arm is received therein.

7. The shielding device of claim 1, further comprising a first entry hole extending through a portion of the wall of the tent portion corresponding to a position of a patient's head in the operative configuration and configured to receive one of an intubation and a ventilation instrument.

8. The shielding device of claim 1, further comprising a second entry hole extending through a portion of the wall of the tent portion, the second entry hole configured to receive an intravenous line therethrough.

9. The shielding device of claim 1, further comprising a suction portion extending through a portion of the wall, the suction port configured to be connected to a suction tube for creating a negative pressure system within the tent portion.

10. The shielding device of claim 1, wherein the flexible material includes a skirt portion which, in the expanded configuration, extends from contacting edges of a wall of the tent portion, the contacting edges configured to contact a surface over which the tent portion is positioned in the operative configuration.

11. The shielding device of claim 1, further comprising a chest compression access area comprising a collapsible material allowing access to a chest of the patient to perform chest compressions.

12. A system for providing a portable barrier between a patient and a healthcare worker, comprising:
a shielding device including a flexible material movable between a folded configuration and an expanded configuration via a tensile frame connected thereto, the tensile frame including a plurality of struts connected to the flexible member, each of the struts biased toward a tensioning configuration for tensioning a portion of the flexible material toward the expanded configuration, in which the flexible material defines a tent portion configured to be positioned over a patient to cover the patient, and each of the struts being deformable toward a collapsed configuration in which portions of the flexible material are folded against itself in the folded configuration; and
a packaging sized and shaped to receive the shielding device therewithin, in the folded configuration, the struts constrained toward the collapsed configuration via an interior surface of the packaging when the shielding device is received therewithin and reverting to the tensioning configuration to move the flexible material toward the expanded configuration as the shielding device is removed from within the packaging.

13. The system of claim 12, wherein the interior space defined via the tent portion is substantially rectangular.

14. The system of claim 13, wherein the tent portion is defined via an anterior surface, a pair of lateral surfaces extending from lateral edges of the anterior surface and a superior surface connecting superior edges of the anterior surface.

15. The system of claim 14, each of the anterior surface, the lateral surfaces and the super surface including one or more deformable struts extending along peripheral edges thereof

16. The system of claim 15, wherein the shielding device further includes a pair of aim holes extending through the superior surface of the tent portion.

17. The system of claim 16, wherein the shielding device includes a pair of sleeves, each of the sleeves extending into the interior space defined via the tent portion from a corresponding one of the arm holes to a ribbed end configured to seal the interior space from an exterior of the of the tent portion when an arm is received therein.

18. The system of claim 12, wherein the shielding device includes a first entry hole extending through a portion of the tent portion for receiving one of an intubation and a ventilation instrument.

19. The system of claim 12, wherein the shielding device includes a second entry hole extending through a portion of the tent portion for receiving an intravenous line therethrough.

20. The system of claim 12, wherein the shielding device includes a suction portion extending through a portion of the tent portion configured to connect a suction tube to the tent portion for providing a negative pressure to an interior space defined via the tent portion.

21. The system of claim 12, wherein the shielding device includes a plurality of clips configured to affix the tent portion to one of a hospital bed, stretcher and gurney in an operative configuration.
